(19) United States
(12) Patent Application Publication
(10) Pub. No.: US 2012/0156787 A1
(43) Pub. Date: Jun. 21, 2012

(54) Laboratory Testing Procedure to Select Acid or Proppant Fracturing Stimulation Treatment for a Given Carbonate Formation

(75) Inventors: Hazim Hussein Abass, Dhahran (SA); Abdulrahman Abdulaziz Al-Mulhem, Dhahran (SA); Miarjuddin R. Khan, Dhahran (SA)

(73) Assignee: Saudi Arabian Oil Company, Dhahran (SA)

(21) Appl. No.: 12/968,806

(22) Filed: Dec. 15, 2010

(51) Int. Cl. G01N 33/24

(52) U.S. Cl. 436/2

(57) ABSTRACT

Embodiments of the present invention enables users to determine the efficiency of acid fracturing in stimulating a formation. The testing procedures of embodiments of the present invention examine the elastic, plastic, and creeping effects on closing an acidized fracture during the life span of an oil/gas well. If it is determined that an acidized fracture will be closed for a given stress and temperature, then proppant fracturing should be used; otherwise, acid fracturing is the stimulation treatment to consider. The testing results also provide an estimation of the lifetime of an acid fracture for a given set of in-situ conditions of stress and temperature. If the lifetime is determined to be too short to make the fracturing treatment economically feasible, a different stimulation method should be considered, such as proppant fracturing or matrix acidizing.

### BACKGROUND OF THE INVENTION

1. Field of the Invention

The present invention related to methods that enable users to determine which type of stimulation application to use in a particular formation without the need for extensive field trials.

2. Description of the Related Art

Various types of fracturing applications can be used in subterranean formations. Acid fracturing is performed to improve well productivity in acid-soluble formations, such as limestone and dolomite. Hydrochloric acid is generally used to create an etched fracture, which upon shut-in, the rough fracture faces close leaving channels that represent fracture conductivity. Proppant fracturing is another stimulation option that has been applied in carbonate formations when acid fracturing is not considered an appropriate treatment. Experience shows that in some carbonate formations, proppant fracturing yields better results than acid fracturing. In other areas, however, acid fracturing is more attractive for economical reasons. Unfortunately, it is difficult to determine which type of fracturing application to use without extensive field trials.

A need exists for methods that would enable operators to determine which type of fracturing application to use with a particular formation without the need to conduct field trials. It would be advantageous if the methods were cost effective and could be performed in a relatively short time period.

### SUMMARY OF THE INVENTION

In view of the foregoing, embodiments of the present invention relate to experimental methods that can be used to determine whether acid fracturing or proppant fracturing would be most efficient for a particular formation.

Embodiments of the present invention enables users to determine the efficiency of acid fracturing in stimulating a given carbonate formation. The testing procedures of embodiments of the present invention examine the elastic, plastic, and creeping effects on closing an acidized fracture during the life span of an oil/gas well. If it is determined that an acidized fracture will be closed for a given stress and temperature, then proppant fracturing should be used; otherwise, acid fracturing is the stimulation treatment to consider. The methods used in embodiments of the present invention evaluate the total effects of elastic, plastic, and creeping properties on fracture closure in carbonate formations following an acid fracturing treatment. The testing results also provide an estimation of the lifetime of an acid fracture for a given set of in-situ conditions of stress and temperature. If the lifetime is determined to be too short to make the fracturing treatment economically feasible, a different stimulation method should be considered, such as proppant fracturing or matrix acidizing.

As part of the total effects of the elastic, plastic, and creeping properties, a creeping test is provided in embodiments of the present invention to study rock deformation under constant stress as a function of time. This test feature simulates the in-situ reservoir conditions where a fracture is exposed to the effective minimum horizontal stress. A typical test involves creation of etched fracture then loading the sample at various stress levels that covers the reservoir stress path during the life of a given well. Flow phase tests are then conducted to examine fracture conductivity as a function of time. The creeping results (fracture closure with time) and flow results data can be analyzed to predict fracture performance with respect to time. The optimum stimulation treatment can be determined without having to go into a field trial.

As an embodiment of the present invention, a method of determining an effective treatment application for a subterranean formation without field trials is provided. In this embodiment, two samples are prepared and then loaded at various stress levels that simulate a reservoir stress path during a life of a given well to obtain creeping results. Flow phase tests are then conducted on the samples to examine a fracture conductivity following the creeping phase to obtain flow phase test results. The creeping results and the flow phase test results are compared for the two samples to determine which sample has a greatest production rate to select an optimum stimulation treatment without field trials.

As another embodiment of the present invention, a method of determining an effective treatment application for a subterranean formation is provided. In this embodiment, the method includes comparing a creeping effect on closing an acidized fracture so that if creeping is sufficient to close the acidized fracture at a predetermined stress and temperature, then proppant fracturing is recommended; and if not, then acid fracturing is recommended.

### BRIEF DESCRIPTION OF THE DRAWING

So that the manner in which the above-recited features, aspects and advantages of the invention, as well as others that will become apparent, are attained and can be understood in detail, more particular description of the invention briefly summarized above can be had by reference to the embodiments thereof that are illustrated in the drawings that form a part of this specification. It is to be noted, however, that the appended drawings illustrate some embodiments of the invention and are, therefore, not to be considered limiting of the invention's scope, for the invention can admit to other equally effective embodiments.

FIG. 1 is a chart illustrating the stress-strain relationship for the examples in accordance with embodiments of the present invention;

FIG. 2 is a chart illustrating the strain behavior for three cycles of loading showing elastic behavior and time dependent creeping for the examples in accordance with embodiments of the present invention;

FIG. 3 is a chart illustrating the stress-strain relationship for the examples in accordance with embodiments of the present invention;

FIG. 4 is a chart illustrating a linear fit of the creeping test at 4000 psi (27.579 MPa), 250° F. (121° C.) for Sample No. 2 in accordance with embodiments of the present invention;

FIG. 5 is a chart illustrating a linear fit of the creeping test at 6000 psi (41.4 MPa), 250° F. (121° C.) for Sample No. 2 in accordance with embodiments of the present invention;

FIG. 6 is a chart illustrating a linear fit of the creeping test at 8000 psi (55.2 MPa), 250° F. (121° C.) for Sample No. 2 in accordance with embodiments of the present invention;

FIG. 7 is a chart illustrating the creeping prediction for the sample in the examples at 4000 psi (27.579 MPa) stress in accordance with embodiments of the present invention;

FIG. 8 is a chart illustrating the creeping prediction of strain versus 1/time for the sample in the examples at 4000 psi (27.579 MPa) stress in accordance with embodiments of the present invention;

FIG. 9 is a chart illustrating the creeping prediction of strain versus log (time) for the sample in the examples at 4000 psi (27.579 MPa) stress in accordance with embodiments of the present invention;

FIG. 10 is a chart illustrating the creep prediction in uniaxial compression of 4000 psi (27.579 MPa) using Burgers model and experimental data in accordance with embodiments of the present invention;

FIG. 11 is a chart illustrating the viscosity of mineral oil that was used as a confining liquid and flowing fluid to evaluate fracture conductivity in the examples in accordance with embodiments of the present invention;

FIG. 12 is a chart illustrating the elastic effect on fracture conductivity as vertical stress was increased from 1000 psi (6.9 MPa) to 4000 psi (27.579 MPa) and the creeping effect at 4000 psi (27.579 MPa) for the examples in accordance with embodiments of the present invention;

FIG. 13 is a chart illustrating the time dependent creeping effect on the production rate at 4000 psi (27.579 MPa) stress for the examples in accordance with embodiments of the present invention;

FIG. 14 is a chart illustrating the effect of creeping on the production rate for different axial stresses for the examples in accordance with embodiments of the present invention;

FIG. 15 is a chart illustrating the elastic effect on the production rate as a function of increasing and decreasing stress for the examples in accordance with embodiments of the present invention;

FIG. 16 is a chart illustrating the elastic effect on the production rate as the axial stress is increased by 100 psi (0.7 MPa) increments for the examples in accordance with embodiments of the present invention;

FIG. 17 is a chart illustrating the decrease in fracture conductivity due to stress increase for the examples in accordance with embodiments of the present invention;

FIG. 18 is a chart illustrating the propped fracture strain behavior that indicates stability with time as the stress is increased for the examples in accordance with embodiments of the present invention;

FIG. 19 is a chart illustrating the effect of vertical stress on the propped fracture strain for the examples in accordance with embodiments of the present invention, which indicates that the production rate is stabilized as the stress is increased;

FIG. 20 is a chart illustrating the time dependent effect of creeping on the production rate of a propped fracture at 5000 psi (34.5 MPa) axial stress for the examples in accordance with embodiments of the present invention;

FIG. 21 is a chart illustrating the effect of acid contact time on fracture conductivity for limestone in accordance with prior art embodiments; and

FIG. 22 is a chart illustrating the effect of acid contact time on fracture conductivity for dolomite in accordance with prior art embodiments.

### DETAILED DESCRIPTION OF THE DISCLOSURE

Hydraulic fracturing using acid and/or proppant is a stimulation technique commonly applied to increase the productivity of hydrocarbon fluids from subterranean formations, such as carbonate formations. Acid fracturing involves injection of an acid solution, such as hydrochloric acid, while proppant fracturing involves injecting a proppant-loaded gel into the formation at a fracturing pressure to propagate an induced fracture. In acid fracturing treatments, the acid creates an etched fracture face with asperities. While in proppant fracturing treatments, the proppant “props” the created fracture to prevent the fracturing from closing. In both types of treatments, a conductive fracture is introduced to the formation to increase well productivity.

Acid fracturing treatments may not always be successful. For example, acid fracturing treatments can fail because the fracture conductivity is not maintained due to fracture closure over time, or the fracture width is small and does not have vertical or lateral continuity, or the created fracture did not penetrate deep enough in the reservoir due to high acid solubility, especially at high temperatures and existence of natural fractures causing the acid to be lost near the wellbore because of high leakoff.

Extensive field trials are typically used to select between two main stimulation treatments, acid fracturing or proppant fracturing, for carbonate formations. Embodiments of the present invention provide for an experimental procedure using full core samples to simulate a fracture behavior following an acid or proppant fracturing treatment. The testing procedure embodiments of the present invention examine the creeping effect on closing an acidized fracture during the life span of an oil/gas well. If creeping is sufficient to close the acidized fracture for a given stress and temperature, then proppant fracturing should be used, otherwise acid fracturing is the stimulation treatment to consider. For purposes of this application, “creeping” generally refers to the tendency of a formation to slowly move or deform permanently under the influence of stresses.

The testing procedure embodiments of the present invention combine and compare the total effects of elastic, plastic, and creeping properties on fracture closure in carbonate formations, following an acid fracturing treatment The testing results also provide an estimation of the lifetime of an acid fracture for a given set of in-situ conditions of stress and temperature. If the lifetime is determined to be too short to make the fracturing treatment economically feasible, a different stimulation method should be considered, such as proppant fracturing or matrix acidizing. Embodiments of the present invention should provide operators with an additional factor for stimulation selection criterion for carbonate formations based on fracture closure characteristics, in addition to acid solubility.

As part of the total effects of the elastic, plastic, and creeping properties, a creeping test is provided in embodiments of the present invention to study rock deformation under constant stress as a function of time. This test feature simulates the in-situ reservoir conditions where a fracture is exposed to the effective minimum horizontal stress. A typical test involves creation of etched fracture then loading the sample at various stress levels that covers the reservoir stress path during the life of a given well. Flow phase tests are then conducted to examine fracture conductivity as a function of time. The creeping results (fracture closure with time) and flow results data can be analyzed to predict fracture performance with respect to time. The optimum stimulation treatment can be determined without having to go into a field trial.

Embodiments of the present invention provide methods that can be used to help decide between one of the major stimulation treatments, specifically whether or not proppant or acid should be used in stimulating carbonate formations. A creeping test is introduced to evaluate the strength of generated asperities as a function of time under constant applied stress.

When deciding to stimulate a carbonate formation, matrix acidizing or acid fracturing is usually the first application that is considered because of the high solubility of carbonate in acid. Acid solubility is typically the primary criterion being used by the industry to base the decision of selecting acidizing as a stimulation treatment for production increase in these formations. Once an acid fracturing treatment starts, it may take a long time to realize that acid fracturing is not suitable for a given carbonate reservoir and proppant fracturing is the treatment that should have been used. Using acid fracturing when proppant fracturing should have been used is a waste of time and money. Time and money could be saved if the formation could have been tested to determine if the acid fracture treatment was appropriate. Embodiments of the present invention can be used to prevent this type of problem from occurring.

Embodiments of the present invention include a laboratory test that can be used to simulate downhole conditions and determine the time-dependant fracture closure of an acid fractured sample. If the test shows that an acid fracture will close in short time as compared to an expected life of a given well, then proppant fracturing is recommended. The methods of the present invention provide selection criteria that is helpful when trying to decide what would be the most successful method to stimulate a carbonate formation.

Embodiments of the present invention are lab based methods that save a great deal of resources that typically would be required when stimulating a carbonate formation. Embodiments of the present invention will help operators make a choice between proceeding with a successful acid fracturing treatment or proceeding with an acid fracturing treatment that will eventually be useless.

As an embodiment of the present invention, a method of determining an effective treatment application for a subterranean formation without field trials is provided. As indicated previously, the methods of the present invention can help users determine whether to use acid fracturing or proppant fracturing for a particular subterranean formation. In this embodiment, two samples are prepared and then loaded at various stress levels that simulate a reservoir stress path during a life of a given well to obtain creeping results. One sample is prepared to simulate acid fracturing, while the other sample is prepared to simulate proppant fracturing. Flow phase tests are then conducted on the samples to examine a fracture conductivity following the creeping phase to obtain flow phase test results. The creeping results and the flow phase test results are compared for the two samples to determine which sample has a greatest production rate to select an optimum stimulation treatment without field trials.

To prepare the acid simulated sample, a hole is created in a center of the sample. The sample is cut horizontally into two portions to simulate a fracture. A surface of the sample is then texturized and exposed to an acid. The two portions of the sample are then bound back together to form the acid simulated sample.

To prepare the proppant simulated sample, a hole is created in a center of the sample.

The sample is cut horizontally into two portions to simulate a fracture. A surface of the sample is then texturized and proppant is applied to the surface of the sample. The two portions of the sample are then bound back together to form the proppant simulated sample.

To load the samples at various stress levels, vertical stress is applied perpendicular to the simulated fracture to simulate a minimum horizontal stress. A vertical strain is then measured at a predetermined stress and time. An external pressure is measured using a confining fluid. A wellbore pressure, a temperature, and a production rate are measured.

The stress and strain levels can vary when loading the samples in embodiments of the present invention. For example, in an aspect, the vertical stress can range from about 2000 psi (13.79 MPa) to about 8000 psi (55.2 MPa). In an aspect, the vertical strain ranges from about 0.00064 in/in (0.16 in/in/psi) to about 0.00126152 in/in. Other suitable stress and strain values that can be used in embodiments of the present invention will be apparent to those of skill in the art and are to be considered within the scope of the present invention.

The methods of the present invention can be used in various types of subterranean formations. In an aspect, the subterranean formation is a carbonate formation. Other suitable types of formations in which the methods of the present invention can be used will be apparent to those of skill in the art and are to be considered within the scope of the present invention.

As another embodiment of the present invention, a method of determining an effective treatment application for a subterranean formation is provided. In this embodiment, the method includes comparing a creeping effect on closing an acidized fracture so that if creeping is sufficient to close the acidized fracture at a predetermined stress and temperature, then proppant fracturing is recommended; and if not, then acid fracturing is recommended.

In embodiments of the present invention, determining the creeping effect comprises the steps of preparing two samples and loading the samples at various stress levels that cover the reservoir stress path during the life of a given well to obtain creeping results. Flow phase tests are then conducted to examine the fracture conductivity following the creeping phase. The creeping results and the flow phase results are then compared for the two samples to determine which sample has the greatest production rate to select an optimum stimulation treatment without field trials.

### Example

Several whole core samples having a 4″ diameter and being approximately 8″ in length were obtained from a well. A ¼″ diameter hole was drilled axially in the center of each sample to allow for a radial flux that can be established through rock matrix or an induced fracture. Each sample was then cut horizontally into two pieces to simulate a fracture. The surfaces simulating a fracture were surface grounded and exposed statically to 15 wt. % acid from both sides either by dipping the sample in acid or placing acid on the surface until no more chemical reaction is observed.

Special care was exercised around the wellbore and the sample external boundary to prevent losing sample contact due to excessive etching at these boundaries. The sample was bound together again with the same alignment before acidizing by matching two marked lines drawn on the sample before cutting. A screen with two screw clamps was mounted on the sample to put it together and provide flow entrance for the confining fluid to flow radially through the simulated etched fracture to the wellbore. The final geometry of the simulated experiment is a vertical wellbore with horizontal fracture. In the case of a propped fracture, the same experimental modeling was applied; however, when the surfaces simulating a fracture were surface grounded, a one layer proppant was placed on one surface. The proppant size is 12/20 mesh with concentration of 0.365 lb/ft².

The sample was then subjected to various conditions to obtain readings of several process conditions. The sample was then positioned inside the rock mechanics loading frame that provide the following measured parameters:
a. Vertical stress applied perpendicular to the fracture that simulated the minimum horizontal stress.
b. Vertical strain was measured from two linear variable differential transformers (LVDTs) that measure the axial strain for a given stress and time.
c. External pressure was measured using the confining fluid. However, since the sample was not jacketed, this fluid was also the reservoir fluid that provided reservoir pressure.
d. Wellbore pressure that was basically atmospheric pressure when the well was put on production.
e. Temperature that was set within the loading frame.
f. Production rate was measured by timing a given production volume.

A creeping test was designed by applying in-situ conditions of temperature and stress for a given sample. Progressive loads simulating stress path exposed on a fracture during production were applied and maintained constant as the resulting deformation was measured. Fracture flow capacity test was conducted under applied progressive stresses to determine the decrease of fracture conductivity due to the elastic, plastic, and viscous effects.

Table 1 lists the samples that were selected for the Example and their perspective depths.

TABLE 1
Core Samples from Well

| Sample No. | Core No. | Tray No. | Sample Depth, ft. |
|---|---|---|---|
| 1 | 1 | 12 | 10623.8-10624.4 |
| 2 | 2 | 20 | 11196.5-11197.0 |
| 3 | 2 | 21 | 11194.4-11194.9 |
| 4 | 2 | 14 | 11214.3-11215.3 |
| 5 | 3 | 3 | 11304.0-11305.6 |
| 6 | 4 | 21 | 11315.3-11315.8 |
| 7 | 2 | 9 | 11229.2-11229.8 |
| 8 | 1 | 16 | 10607.6-10608.2 |

The chemistry of the carbonates contained in the well was evaluated by x-ray diffraction (XRD) and is shown in Tables 2 and 3.

TABLE 2
Sample 1 XRD Analysis

| Compounds | Wt. % |
|---|---|
| Anhydrite-CaSO₄ | 61 |
| Quartz-SiO₂ | <1 |
| Calcite-CaCO₃ | — |
| Dolomite-CaMg(CO₃)₂ | 39 |
| Magnetite-Fe₃O₄ | — |
| Kaolinite-Al₂Si₂O₅(OH)₄ | — |
| Albite-NaAlSi₃O₈ | <1 |
| Illite/muscovite | — |
| Microcline-K AlSi₃O₈ | — |

TABLE 3
Sample 2 XRD Analysis

| Compounds | Sample 2A, wt. % | Sample 2B, wt. % |
|---|---|---|
| Dolomite-CaMg(CO₃)₂ | 67 | 3 |
| Anhydrite-CaSO₄ | 31 | 2 |
| Calcite-CaCO₃ | 1 | 95 |
| Quartz-SiO₂ | 1 | Trace |

To establish a baseline for fracture flow evaluation each sample was tested before drilling a wellbore and creating a simulated fracture. The static Young's modulus was measured by applying confining pressure around the sample, and then increasing the axial compressive stress until a nonlinear portion of the stress-strain relationship was established for a given confining pressure. A flow test was performed with a wellbore drilled in the center of the sample and no flow was observed through the rock matrix.

Creeping Test of Acid-Fracture Samples

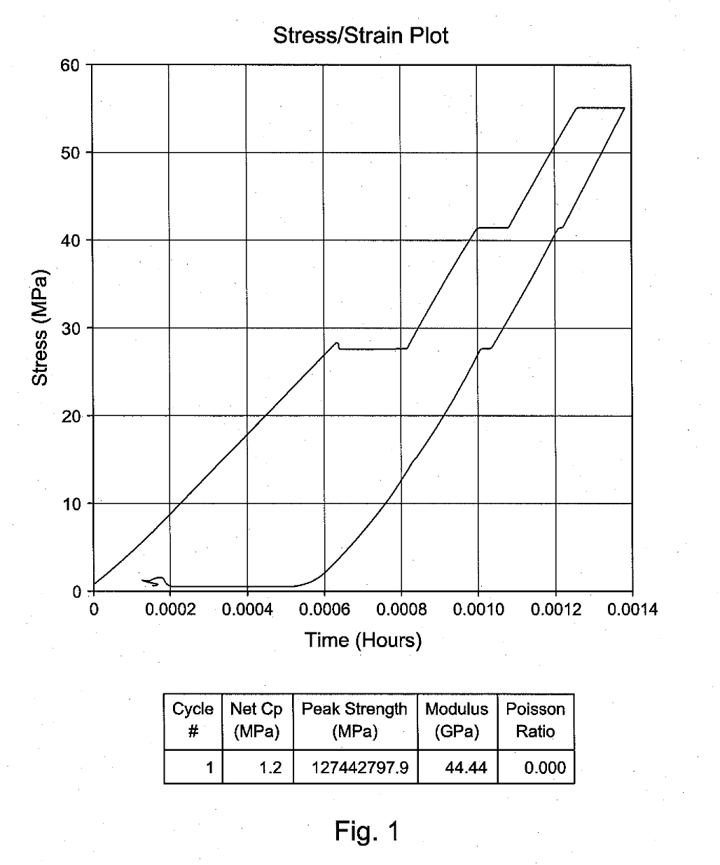

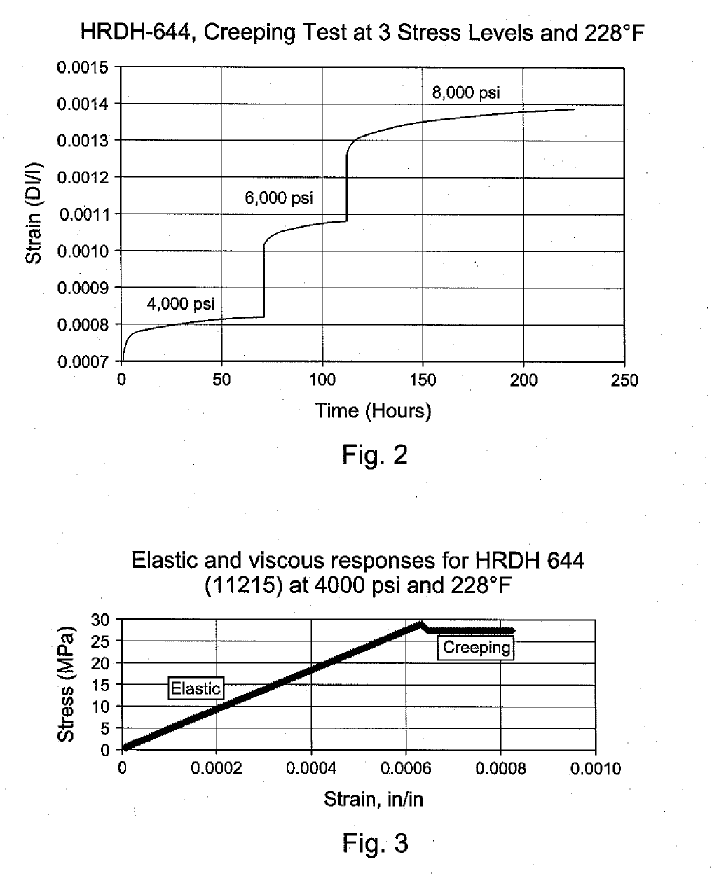

A creeping test was designed to study rock deformation under constant stress as a function of time. This test simulated the in-situ reservoir conditions where a fracture is exposed to the effective minimum horizontal stress. A typical test involved loading a sample at three stresses: 4000 psi (27.579 MPa), 6000 psi (41.4 MPa), and 8000 psi (55.2 MPa). FIG. 1 shows the stress-strain relationship for these three stress steps. Each step included the elastic and viscous responses of the sample for a given stress. The first step showed that the elastic strain for loading the sample from 0 psi (0 MPa) until 4000 psi (27,579 MPa) resulted in the maximum strain being about 0.00064 in/in or (0.16 in/in/psi). This strain value is equivalent to a Young's modulus of 6.25×10⁶ psi (43092 MPa). This elastic response is represented in FIG. 2 as the immediate increase in strain which is not time dependent. The stress was then maintained constant at 4000 psi (27.579 MPa) for 71.19 hours to obtain the creeping characteristics for the sample. The creeping profile suggests that the sample exhibited the primary and secondary creeping phases but had not shown any sign of tertiary creeping, which was expected for such a high Young's modulus sample. The elastic, primary creeping and secondary creeping phases were shown, but did not show any sign of tertiary creeping, which was expected for such a high Young's modulus sample. The elastic, primary creeping and secondary creeping responses can be observed in FIG. 2 as stress-time representation, while FIG. 3 shows the elastic and creeping responses as a stress-strain representation. The total strain accumulated at the end of 71.19 hours was 0.00082278 in/in (1.15575×10⁻⁶ in/in/hr) as shown in FIG. 2.

The stress was then increased to 6000 psi (41.4 MPa), where the elastic strain increased to 0.00100201 in/in, which means that the elastic strain generated from the additional 2000 psi (13.79 MPa) is 0.0001793. This strain value is equivalent to a Young's modulus of 11.16×10⁶ psi (76946 MPa), which is an indication that the sample has become stronger during the second cycle as micro cracks and natural fractures have been closed. The stress was then kept constant for about 41.31 hours to have a total testing time of 112.5 hours. The total strain at this time was 0.00108228 in/in. The primary and secondary creeping yielded a strain of 0.0008027 in/in (1.9431×10⁻⁶ in/in/hr).

The stress was then increased to 8000 psi (55.2 MPa), where the elastic strain increased to 0.00126152 in/in, which means that the elastic strain generated from the additional 2000 psi (13.79 MPa) is 0.00017924 in/in. This strain value is equivalent to a Young's modulus of 11.16×10⁶ psig (76 945 MPa), which is an indication that the sample has not changed from the last cycle. The stress was then kept constant at 8000 psi (55.2 MPa) for about 118.11 hours to have a total testing time of 230.61 hours. The total strain at this time was 0.00138577 in/in. The primary and secondary creeping yielded a strain of 0.00012425 in/in (0.53879×10⁻⁶ in/in/hr).

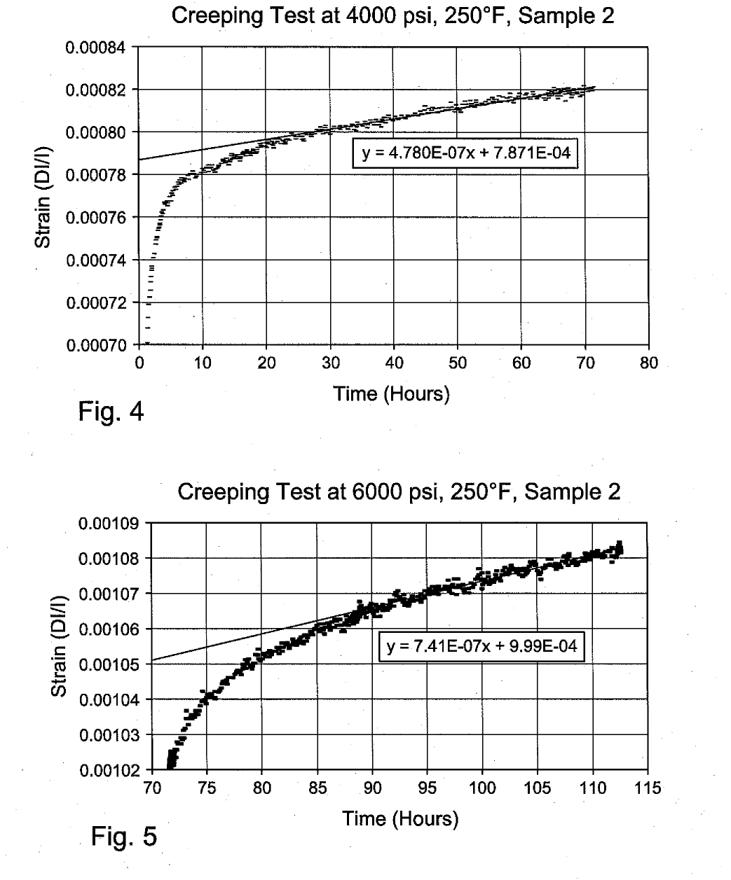

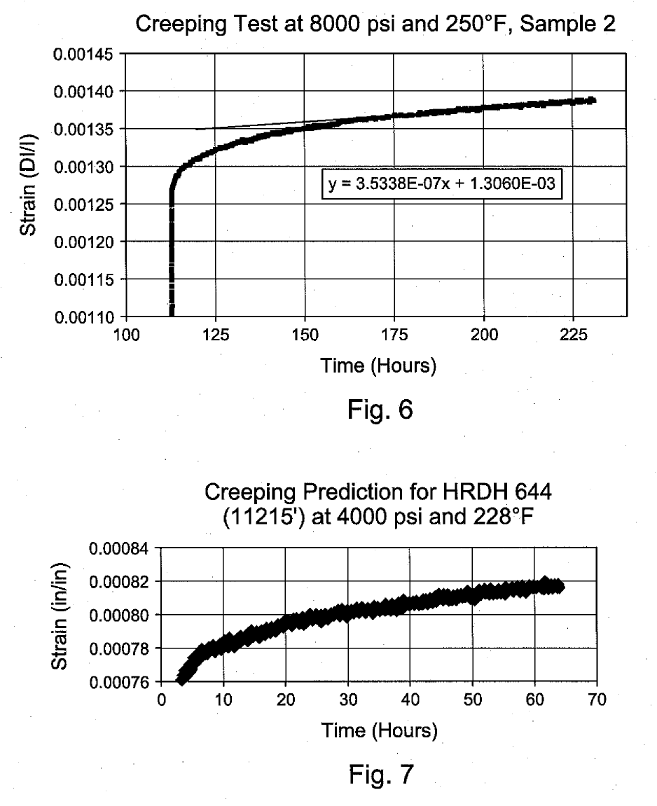

FIGS. 4, 5, and 6 show a linear fitting of the secondary creeping portions for three tests described by the following equations:

ε=4.78×10⁻⁷t+7.87×10⁻⁴ at 4000 psi (27.579 MPa)

ε=4.78×10⁻⁷t+9.99×10⁻⁴ at 6000 psi (41.4 MPa)

ε=3.5338×10⁻⁷t+1.306×10⁻³ at 8000 psi (55.2 MPa)

To predict the creeping strain as a function of time, three time-prediction functions are presented:

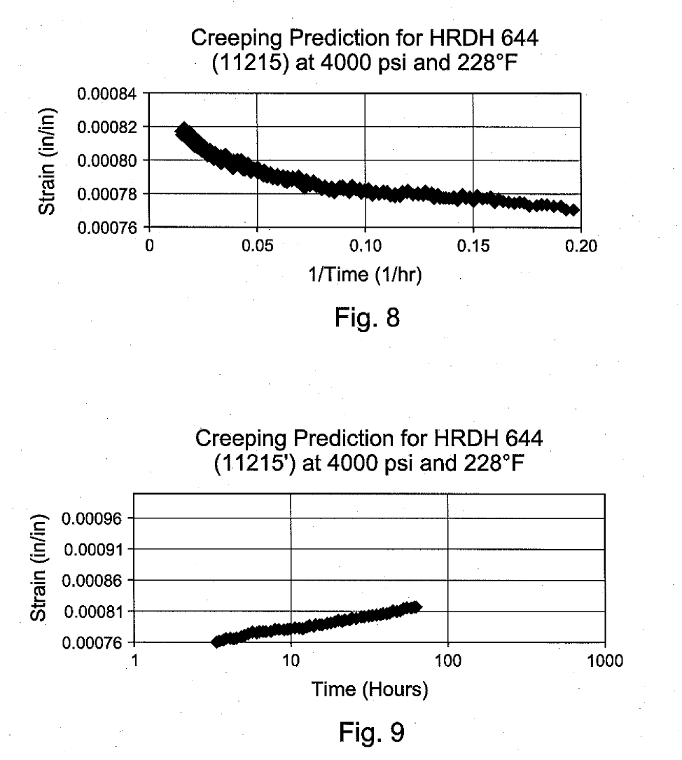

1) Time (t) as shown in FIG. 7,
2) Reciprocal time (lit) as shown in FIG. 8, and
3) Log time (log t) as shown in FIG. 9.

The time function required a linear fitting for the secondary creeping behavior to determine the creeping magnitude at any given time assuming that the creeping will continue at the secondary phase and will not approach a tertiary creeping phase, which was a valid assumption as the rock was very strong and would not exhibit plastic flow.

The reciprocal time function could be used to determine the interception at zero which should present the maximum creeping obtained after a very long time approximated by infinity. This function was not linear and predicting the strain at any time would require an extrapolation of a non-linear function that is mathematically difficult. The log time function, however, provides a mean to extrapolate the cumulative strain for a given time.

Creep Modeling

To model the complete creeping response (primary and secondary), Burgers model was used to describe the axial strain as a function of time for a sample subjected to constant axial stress is given by Goodman, 1980;

$$ɛ ( t ) = \frac{2 σ}{9 K} + \frac{σ}{3 {G}_{2}} + \frac{σ}{3 {G}_{1}} - \frac{σ}{3 {G}_{1}} {e}^{- ( {G}_{1} t / {η}_{1} )} + \frac{σ}{3 {η}_{2}} t$$

where:
K=Bulk modulus (K=E/3(1-2v)), psi
σ=Axial stress, psi
G₂ Elastic shear modulus, psi
G₁=A rock property that controls the amount of delayed elasticity, psi
η₂=The rate of viscous flow, psi·hr
η₁=A parameter that determines the rate of delayed elasticity, psi·hr
t=time, hr

This model includes the instantaneous strain, transient creep, and steady state creep. The experimental creep data for 4000 psi (27.579 MPa) axial stress was matched by Burgers model using the following parameters.

### σ=4000, psi (27.6 MPa)

### K=3.75×10⁶ psi (25855 MPa)

### G₁=16×10⁶ psi (110,316 MPa)

### G₂=2.9×10⁶ psi (19,995 MPa)

η₂=2.2×10⁹, psi·min (15.17×10⁶ MPa·min)
η₁=40×10⁶, psi·min (275,790 MPa·min)
t=time, hr

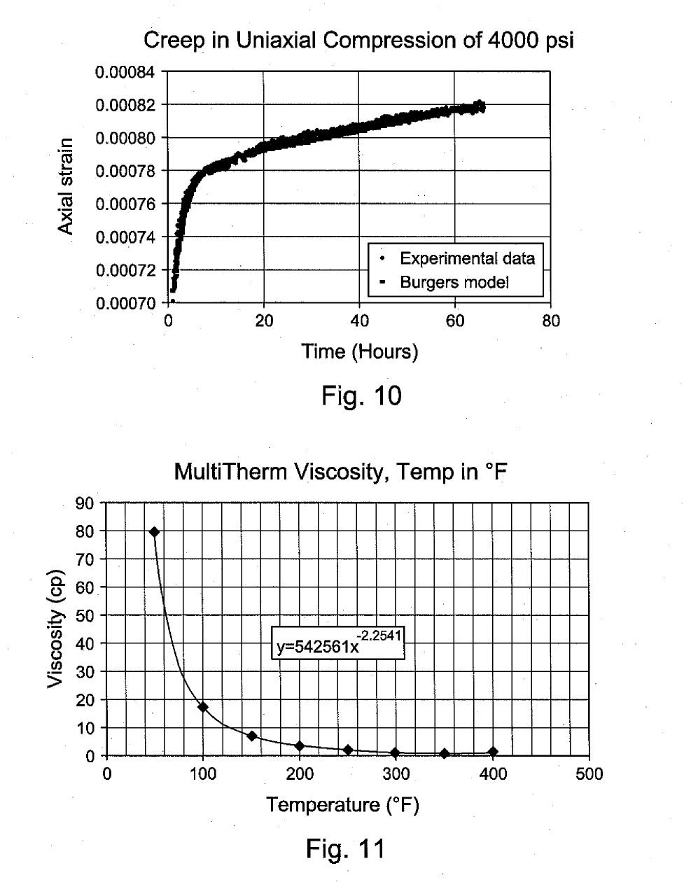

FIG. 10 shows the experimental and model prediction for one of the creeping tests at 4000 psi (27.579 MPa) axial stress. The model clearly illustrates the non-linear behavior of the time-dependant behavior and describes the constitutive behavior of the sample. The model parameters reflect physical properties and can match other experimental data for other axial stress values performed on the same sample. Different samples required adjusting these parameters to match the intrinsic properties of the rock sample because of the heterogeneities of this formation.

The average width of an induced fracture subjected to an applied net pressure, P, is given by Jeager and Cook, 1979:

$${W}_{av} = \frac{mP ( 1 - {v}^{2} ) \sqrt{A}}{E}$$

where A is the area for a fracture and m is a numerical geometry factor ranging from 0.71 to 0.95 depending on a given fracture length. If this equation is compared to simple plain strain equation, the effect of a pressurized fracture in developing fracture width through rock displacement is determined by the factor \sqrt{A}. If we assume a square fracture, the distance through the rock mass perpendicular to the fracture that contributes to fracture displacement is equivalent to fracture height or length, which suggests that the loading affects an equivalent distance to the applied area. This equivalence indicates that there is a critical distance away from the fracture within which the rock mass is deforming, and beyond this region, rock formation does not experience the applied stress. Therefore, the formation beyond the critical distance does not experience the applied stress and, thus, does not exert any elastic rebound deformation toward the fracture upon releasing the pressure in the fracture.

The critical distance can be assumed to contribute to the fracture closure upon removal of the applied fracturing pressure. The strain function presented in FIG. 10 determines the strain at a given time. This strain is basically defined by:

$$Strain = \frac{Δ w}{L}$$

where Δw is the fracture displacement during width development or closure, and L is the critical distance defined above. The critical distance can be mimicked to assume the rock mass contributing to the time-dependent closure including the primary and secondary creeping phases.

Example Calculation

The closure stress in the formation can be estimated from the minimum horizontal stress, pore pressure, and Biot's constant. For the formation, the minimum horizontal stress is 9750 psi (67.2 MPa) based on 0.75 fracture gradient and 13000 ft depth. The reservoir pressure is assumed 65000 psi (448.2 MPa), and Biot's constant of 0.8. The effective closure stress is therefore determined to be 4550 psi (31.4 MPa) based on the following equation:

σ′=σ−αPₚ

where: σ′ is the net effective stress acting on the fracture surface at a given pore pressure, σ is the total in-situ horizontal stress, α is Biot's constant, and Pₚ, is the pore pressure.

Based on the above data, the primary and secondary creeping data are given as:

Elastic response: 0.0007
Primary creeping: 0.0001 in/in for the first 30 hours
Secondary creeping: 4.78×10⁻⁷ in/in/hr at time>30 hours

Assuming a critical distance of 30 ft, the following displacement is obtained:

Elastic response: 0.0007*30*12-0.25 in
Primary creeping: 0.0001*30*12=0.036 in after 30 hours from shut-in
Secondary creeping: 4.78×*30*12=0.0001721 in/hr
=0.3441 in after 2000 hours.

The displacement due to creeping compared to elastic response becomes significant with time as it shows that after 1000 hours it is more than the elastic one. This displacement will not close the fracture directly, but it is manifested into stress applied on the contact points (asperities) in acid fracturing or on the proppant grains of the proppant pack in proppant fracturing. The conductivity of a propped fracture will not suffer much decline as compared to the acid fracture for two reasons:

1) Since a proppant fracture is typically designed to be packed with proppant grains, a single grain will face much less stress than an asperity in an acid fracture.
2) The strength of a spherical proppant grain is much higher than an irregular asperity in an acid fracture.

This feature can be described mathematically as follows:

$${σ}_{c} = \frac{{F}_{n}}{{A}_{c}} = \frac{{σ}_{h}}{{A}_{c} / {A}_{n}}$$

where: σ_{c} is the average stress on a given asperity, Fₙ is the normal force which is equal to the normal stress (minimum horizontal stress) times the normal area, and Aₙ is the normal area. The denominator of the above equation is the ratio of the contact area divided by the normal area. As the effective normal stress (minimum horizontal stress minus the reservoir pressure) increases, some of the asperities fail and therefore this ratio increases.

On the other hand, direct displacement that closes the fracture due to creeping happens within a rock space between two consecutive contact points.

Fracture Conductivity of Acid-Fractured Samples

To evaluate the effect of elastic and creeping displacements on fracture conductivity, flow testing was conducted using the confining fluid that is a type of mineral oil having a viscosity as a function of temperature as shown in FIG. 11. Using the sample configuration as described herein, the rate was measured as a function of time for a given drawdown pressure, axial stress, and time. Fracture conductivity can be calculated from the radial form of Darcy's law as follows:

$$Q = \frac{0.78168 * K * H Δ P}{μ ln \frac{re}{rw}}$$

$$and , \\ KH = \frac{Q μ ln \frac{{r}_{e}}{{r}_{w}}}{0.78168 Δ P}$$

The following data are applied:
rₑ=external radius, 4″
r_{w}=wellbore radius, 0.125″
Pc=external pressure, 725 psi (5 MPa)
Pw=wellbore pressure, 14.7 psi (0.1 MPa)
ΔP=pressure drawdown (Pe−Pw), psi
M=oil viscosity, 37.8 cP at 69.8° F. (21° C.), and 3.1 cP at 212° F. (100° C.).

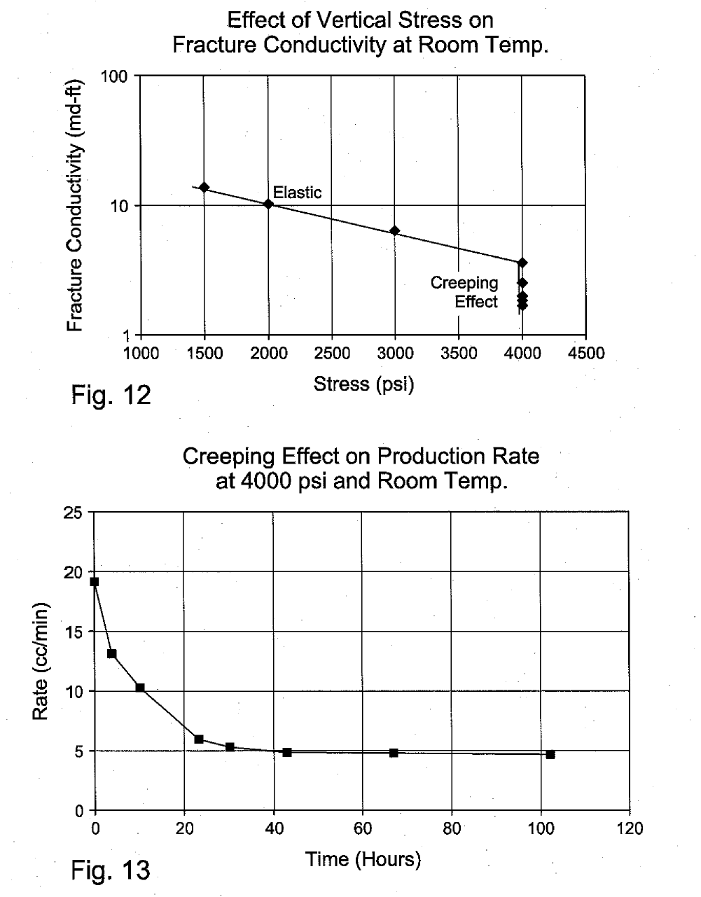

FIG. 12 shows the elastic effect on the production rate at room temperature. The stress was increased in a step-wise function from about 1000 psi (6.9 MPa (to about 4000 psi (27.579 MPa). The production rate decreased from about 180 cc/min to about 20 cc/min. The stress was then maintained at 4000 psi (27.579 MPa) to evaluate the creeping effect as depicted in FIG. 13. The production rate declined from about 20 cc/min to about 5 cc/min after 100 hours.

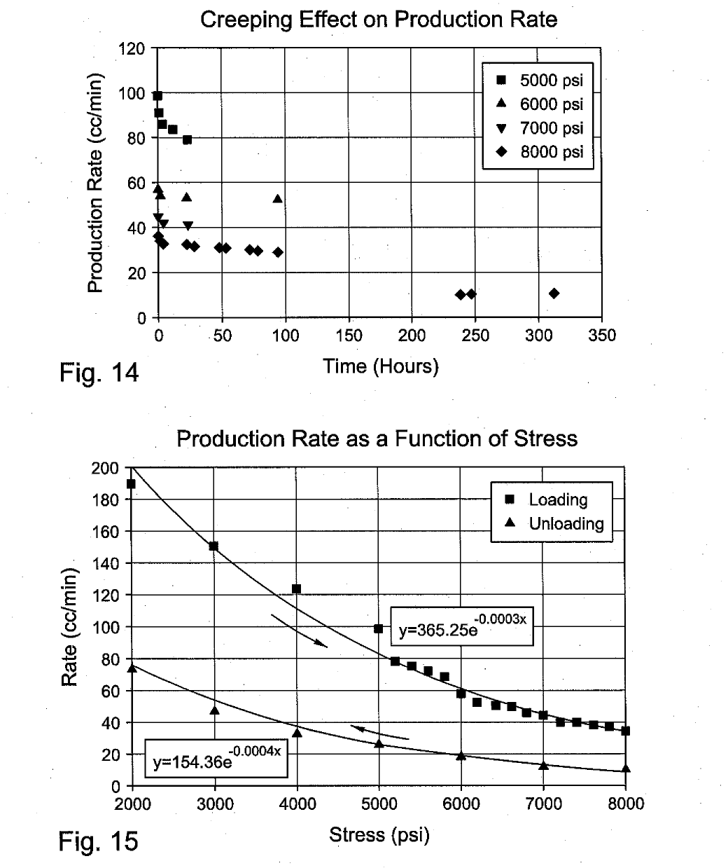

FIG. 14 shows the creeping effect at 5000 psi (34.5 MPa), 6000 psi (41.4 MPa), 7000 psi (48.3 MPa), and 8000 psi (55.2 MPa) axial stresses. All graphs show a decline of the production rate with time which is corresponding to a combination effect that reduces fracture width due to the elastic displacement, plastic failure of the contact points and creeping displacement as described herein.

FIG. 15 shows the loading and unloading effect on the production rate decline as a function of increasing and decreasing stress path respectively. The stress increasing path has closed microfractures and fissures that were created during coring and removal of the confinement condition. The unloading path is more representative to the in-situ conditions found in the reservoir.

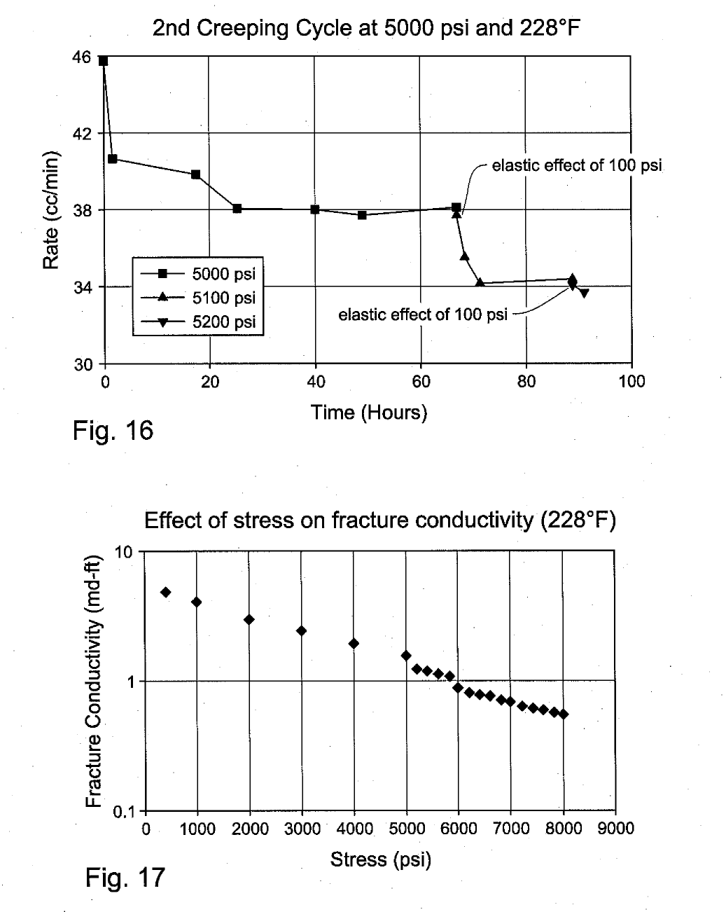

FIG. 16 shows the effect of a small stress increase of 100 psi (0.7 MPa) on production rate which shows a small elastic effect followed by an appreciable decline due to creeping effect. This creeping effect is more representative of plastic flow of the contact points rather than a creeping effect of rock matrix.

FIG. 17 illustrates the calculated fracture conductivity on a log scale as a function of stress.

Creeping Test of Propped-Fracture Samples

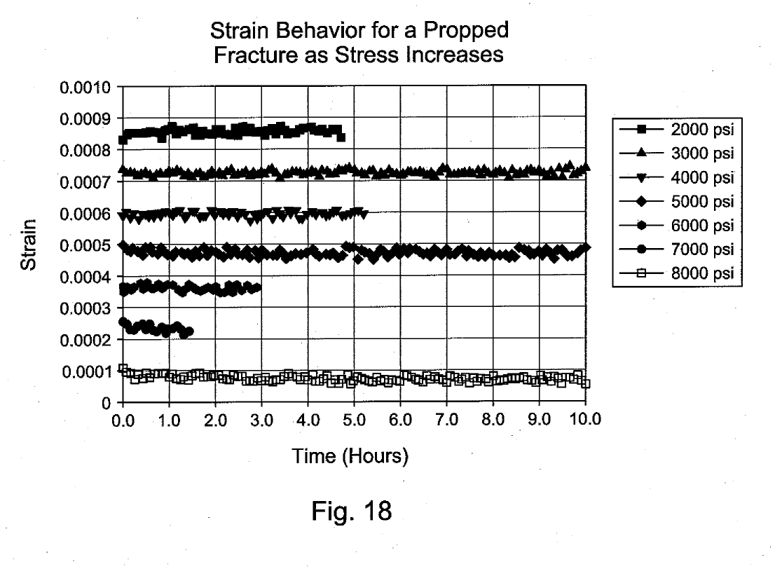

The propped samples were tested at seven stress levels; 2000 psi (13.79 MPa), 3000 psi (20.7 MPa), 4000 psi (27.579 MPa), 5000 psi (34.5 MPa), 6000 psi (41.4 MPa), 7000 psi (48.3 MPa), and 8000 psi (55.2 MPa) as shown in FIG. 18. The strain generated from one stress level to the next progressive one indicated the elastic strain; however, the time dependant strain characterized the creeping behavior, All stress levels showed insignificant creeping strain which suggested that the proppant bed was counteracting the applied stress. Crushing was not being evaluated as there was only one layer of proppant. However embedment was noticed but it was not significant to affect the production rate.

Fracture Conductivity of Propped-Fracture Samples

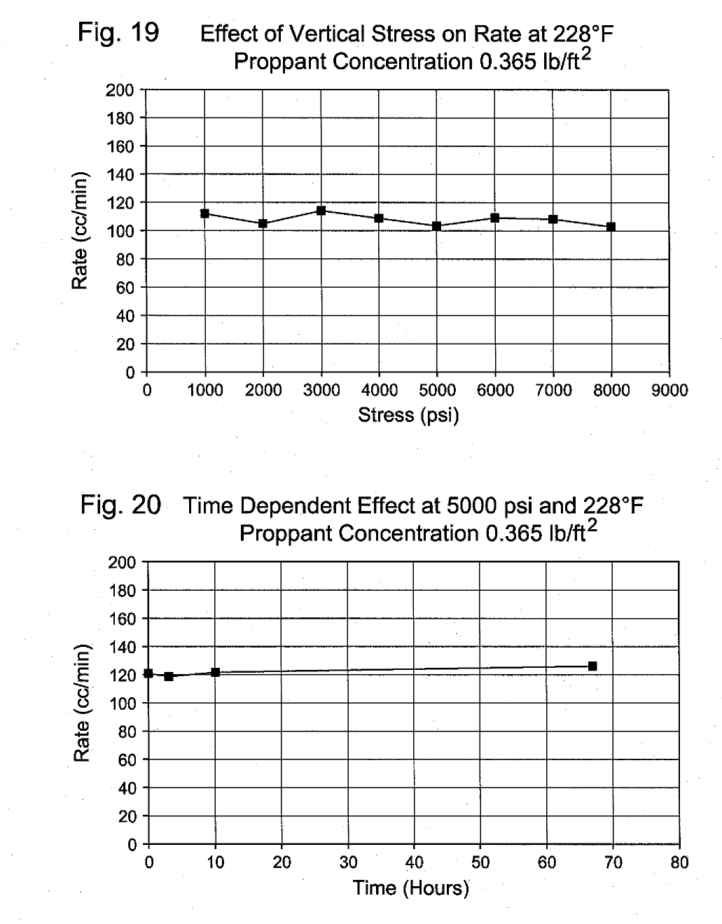

Fracture conductivity was evaluated under different stress levels for the propped-fracture samples. The effect of stress on production rate is shown in FIG. 19. The crushing effect was not well simulated because there was only one layer of proppant. FIG. 19 shows that a propped fracture would sustain productivity while an acid fracture would exhibit drastic decline due to the decrease in fracture width as a function of increasing stress. The effect of creeping on fracture conductivity was shown in FIG. 20, which demonstrates that the effect of creeping on propped-fracture conductivity was not significant.

Other Factors to Consider

In addition to fracture closure, there are other important factors that must be considered to decide on selecting a proppant or acid fracturing treatment. These factors include:
1. Vertical fracture communication
2. Fracture width
3. Fracture length
4. Effect of acid on mechanical strength of a fracture surface
5. Condensate banking
   Each of these factors are described herein.

Vertical Fracture Communication

Rock heterogeneity is essential to create an uneven etched pattern to generate appreciable conductivity. This is true when the heterogeneity is occurring horizontally; however if these heterogeneities occur vertically then the result will be lack of vertical communication along the created fracture. Two types of vertical heterogeneities have been observed, namely different lithologies and horizontal sterilities.

Fracture Width

Fracture width and length varies significantly between acid fracturing and proppant fracturing. Fracture width in acid fracturing is created from the etching mechanism and upon closing, the channels will be left open because of the non-smooth surfaces of the created fracture. In proppant fracturing, the fracture will be closing on a proppant bed leaving a continuous fracture (not channels) connecting the reservoir to a wellbore. Fracture continuity and width will be more evident in proppant fracturing than in acid fracturing.

Fracture Length

Fracture length in acid fracturing and proppant fracturing will be different due to the dissimilar fracture mechanics involved in these techniques. In proppant fracturing, fracturing gel is not reactive with the formation, and therefore can penetrate deeper as compared to acid fracturing for a given fracturing-fluid volume especially at high reservoir temperature. Longer fractures are generally created in proppant fracturing as compared to acid fracturing because of the differences in reactivity of the fracturing fluids.

Effect of Acid on Mechanical Strength of Fracture Surface

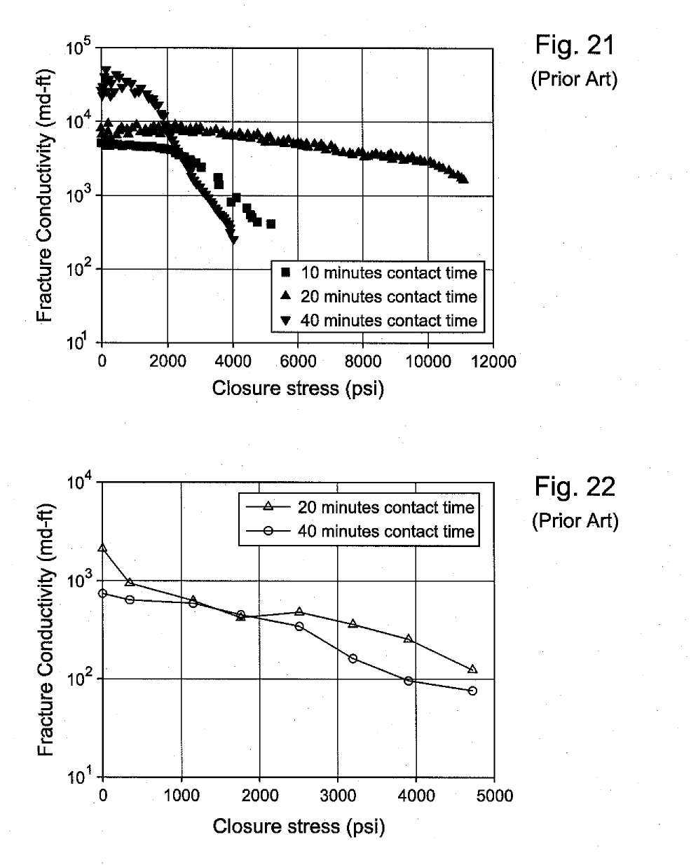

Extending acid contact time may not be beneficial to obtain fracture conductivity as it can weaken the fracture surface and may make it more vulnerable for creeping and compressive failure of the contact points. It has been previously been shown that the conductivity created by 20 minutes of acid contact time was higher than that created by 40 minutes for both dolomite and limestone samples. This difference was even more evident at higher stress as shown in FIGS. 21 and 22. A possible explanation is that acid exposure weakened the rock structure along the fracture surface resulting in greater sensitivity to closure stress. From a rock mechanics point of view, the rock becomes more plastic and the contact points tend to fail and flow at higher closure stress. Additionally these contact points need not to be sharp and long as their failure becomes more apparent. This effect is more pronounced near the wellbore as the acid contact time is the maximum. It is recommended to over-displace the acid well into the fracture to prevent much dissolution near the wellbore.

Nasr-El-Din, et al., 2002, and Rahim et al., 2002, used the embedment strength property to evaluate the effect of acid on surface hardness of core samples from Khuff formations as shown in Table 5. The results indicate an appreciable decrease in surface hardness due to acid reaction. This decrease in hardness weakens the contact points and causes them to fail under the effect of closure stress. Additionally, it creates a more ductile fracture surface that becomes more vulnerable to creeping effect.

TABLE 5
Rock Embedment Strength of Khuff, Zillur, 2002

| | Rock embedment strength | Rock embedment strength |
|---|---|---|
| Lithology | before acidizing, psi (MPa) | after acidizing, psi (MPa) |
| Limestone | 70425 (485.6) | 50784 (350) |
| Limestine | 51072 (352.1) | 31494 (217) |
| Limestone/ | 59041 (407) | 39040 (269) |
| Dolomite | | |
| Dolomite | 62027 (427.7) | 49324 (340) |
| Dolomite | 129988 (896) | 47674 (329) |

Condensate Banking

The effect of condensate banking on productivity is well documented in the literature. The question is whether proppant fracturing can ease the severity of production decline due to condensate banking as compared to acid fracturing. Settari, et al., 1996, using reservoir simulation, showed that proppant fracturing is effective in mitigating the effect of condensate blockage. Condensate banking, however, was not used in embodiments of the present invention.

Although the present invention has been described in detail, it should be understood that various changes, substitutions, and alterations can be made hereupon without departing from the principle and scope of the invention. Accordingly, the scope of the present invention should be determined by the following claims and their appropriate legal equivalents.

The singular forms “a”, “an” and “the” include plural referents, unless the context clearly dictates otherwise.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

Throughout this application, where patents or publications are referenced, the disclosures of these references in their entireties are intended to be incorporated by reference into this application, in order to more fully describe the state of the art to which the invention pertains, except when these reference contradict the statements made herein.

What is claimed is:

1. A method of determining an effective stimulation treatment application for a subterranean formation without field trials, the method comprising the steps of
a. preparing two samples, with one sample being an acid simulated sample and another sample being a proppant simulated sample;
b. loading the samples at various stress levels that simulate a reservoir stress path during a life of a given well to obtain creeping results;
c. conducting flow phase tests on the samples to examine a fracture conductivity following the creeping phase to obtain flow phase test results; and
d. comparing the creeping results and the flow phase test results for the two samples to determine which sample has a greatest production rate to select the effective stimulation treatment application without field trials.

2. The method of claim 1, where the step of creating the acid simulated sample comprises:
a. creating a hole in a center of the sample;
b. horizontally cutting the sample into two portions to simulate a fracture;
c. texturizing a surface of the sample;
d. exposing the surface of the sample to an acid; and
e. binding the two portions back together.

3. The method of claim 1, where the step of creating the proppant simulated sample comprises:
a. creating a hole in a center of the sample;
b. horizontally cutting the sample into two portions to simulate a fracture;
c. texturizing a surface of the sample;
d. applying proppant to the surface of the sample; and
e, binding the two portions back together.

4. The method of claim 1, wherein the step of loading the samples at various stress levels comprises:
a. applying vertical stress perpendicular to the simulated fracture to simulate a minimum horizontal stress;
b. measuring a vertical strain at a predetermined stress and time;
c. measuring an external pressure using a confining fluid; and
d. measuring a wellbore pressure, a temperature, and a production rate.

5. The method of claim 4, wherein the vertical stress ranges from about 2000 psi (13.79 MPa) to about 8000 psi (55.2 MPa).

6. The method of claim 4, wherein the vertical strain ranges from about 0.00064 in/in (0.16 in/in/psi) to about 0.00126152 in/in.

7. The method of claim 1, wherein the subterranean formation is a carbonate formation.

8. A method of determining an effective stimulation treatment application for a subterranean formation, the method comprising the steps of
a. comparing a creeping effect on closing an acidized fracture;
b. if creeping is sufficient to close the acidized fracture at a predetermined stress and temperature, then proppant fracturing is recommended as the effective stimulation treatment application; and
c. if not, then acid fracturing is recommended as the effective stimulation treatment application.

9. The method of claim 8, wherein determining the creeping effect comprises the steps of:
a. preparing two samples;
b. loading the samples at various stress levels that cover the reservoir stress path during the life of a given well to obtain creeping results;
c. conducting flow phase to examine the fracture conductivity following the creeping phase; and
d. comparing the creeping results and the flow phase results for the two samples to determine which sample has the greatest production rate to select an optimum stimulation treatment without field trials.

10. The method of claim 9, wherein the step of preparing two samples comprises:
a. preparing an acid simulated sample; and
b. preparing a proppant simulated sample.

11. The method of claim 10, where the step of creating the acid simulated sample comprises:
a. creating a hole in a center of the sample;
b. horizontally cutting the sample into two portions to simulate a fracture;
c. texturizing a surface of the sample;
d. exposing the surface of the sample to an acid; and
e. binding the two portions back together.

12. The method of claim 10, where the step of creating the proppant simulated sample comprises:
a. creating a hole in a center of the sample;
b. horizontally cutting the sample into two portions to simulate a fracture;
c. texturizing a surface of the sample;
d. applying proppant to the surface of the sample; and
e. binding the two portions back together.

13. The method of claim 9, wherein the step of loading the samples at various stress levels comprises:
a. applying vertical stress perpendicular to the simulated fracture to simulate a minimum horizontal stress;
b. measuring a vertical strain at a predetermined stress and time;
c. measuring an external pressure using a confining fluid; and
d. measuring a wellbore pressure, a temperature, and a production rate.

14. The method of claim 13, wherein the vertical stress ranges from about 2000 psi (13.79 MPa) to about 8000 psi (55.2 MPa).

15. The method of claim 13, wherein the vertical strain ranges from about 0.00064 in/in (0.16 in/in/psi) to about 0.00126152 in/in.

16. The method of claim 9, wherein the subterranean formation is a carbonate formation.

17. A method of determining an effective stimulation treatment application for a subterranean formation without field trials, the method comprising the steps of:
a. creating a hole in a center of two core samples;
b. horizontally cutting each core sample into two portions to simulate a fracture;
c. texturizing a surface of each sample;
d. exposing the surface of one sample to an acid;
e. applying proppant to the surface of another sample;
f. binding each of the portions of each sample back together to prepare an acid simulated sample and a proppant simulated sample;
g. loading the samples at various stress levels that simulate a reservoir stress path during a life of a given well to obtain creeping results, the stress levels ranging from about 2000 psi (13.79 MPa) to about 8000 psi (55.2 MPa);
h. conducting flow phase tests on the samples to examine a fracture conductivity following the creeping phase to obtain flow phase test results; and
i. comparing the creeping results and the flow phase test results for the two samples to determine which sample has a greatest production rate to select the effective stimulation treatment application without field trials.

18. The method of claim 17, wherein the step of loading the samples at various stress levels comprises:
a. applying vertical stress perpendicular to the simulated fracture to simulate a minimum horizontal stress;
b. measuring a vertical strain at a predetermined stress and time;
c. measuring an external pressure using a confining fluid; and
d. measuring a wellbore pressure, a temperature, and a production rate.

19. The method of claim 17, wherein the subterranean formation is a carbonate formation.
